(19) United States
(12) Patent Application Publication
(10) Pub. No.: US 2021/0128536 A1
(43) Pub. Date: May 6, 2021

(54) WEAKLY BASIC DRUG AND IONIC POLYMER PHARMACEUTICAL FORMULATIONS AND METHODS OF FORMATION AND ADMINISTRATION THEREOF

(71) Applicants: DISPERSOL TECHNOLOGIES, LLC, Georgetown, TX (US); BOARD OF REGENTS, THE UNIVERSITY OF TEXAS SYSTEM, Austin, TX (US)

(72) Inventors: Robert O. WILLIAMS, III, Austin, TX (US); Scott V. JERMAIN, Georgetown, TX (US); Daniel Alan DAVIS, Austin, TX (US); Dave A. MILLER, Georgetown, TX (US)

(73) Assignees: DISPERSOL TECHNOLOGIES, LLC, Georgetown, TX (US); BOARD OF REGENTS, THE UNIVERSITY OF TEXAS SYSTEM, Austin, TX (US)

(21) Appl. No.: 17/065,664

(22) Filed: Oct. 8, 2020

Related U.S. Application Data
(60) Provisional application No. 62/929,212, filed on Nov. 1, 2019.

(51) Int. Cl. A61K 31/444; A61K 47/38; A61K 47/32; A61K 9/16; A61K 9/00; A61K 31/496; A61K 31/136; A61K 31/704; A61K 31/475; A61K 31/167; A61K 31/5415

(52) U.S. Cl. CPC A61K 31/444; A61K 47/38; A61K 47/32; A61K 9/1652; A61K 9/0053; A61K 31/5415; A61K 31/136; A61K 31/704; A61K 31/475; A61K 31/167; A61K 31/496

(57) ABSTRACT

The present disclosure relates to pharmaceutical formulations including a weakly basic drug and an ionic polymer in an amorphous solid dispersion, as well as methods of forming such pharmaceutical formulations, and methods of administering such pharmaceutical formulations.

### PRIORITY CLAIM

This application claims benefit of priority to U.S. Provisional Application Ser. No. 62/929,212, filed Nov. 1, 2019, the entire contents of which are hereby incorporated by reference.

### TECHNICAL FIELD

The present disclosure relates to amorphous solid dispersion pharmaceutical formulations and methods of forming and administering such pharmaceutical formulations.

### BACKGROUND

Drugs that have poor solubility in water create many limitations that make it difficult for those drugs to be delivered successfully into the body. One of these limitations is delivering low solubility drugs that are weakly basic and that have low solubility in water to the lower intestine. This limitation occurs when poorly water-soluble drugs that are weakly basic are formulated as an immediate release dosage form. Weakly basic drugs are more soluble in the acidic media of the stomach and less soluble in the pH environment of the small intestine (typically pH≥5) following conversion to their unionized form (pH>pKa). Therefore, transit of weakly basic drugs from the stomach into the upper small intestine results in precipitation depending on the drug solubility as a function of pH. When supersaturated drug levels are obtained in gastric media with a weakly basic drug, severe drug precipitation has been shown after pH transition in small intestinal pH during in vitro dissolution. Ultimately, low and variable bioavailability occurs, because the upper small intestine typically constitutes the principle site of absorption. Weakly basic drugs also have a limitation when formulated as a modified release dosage form (e.g., delayed release); The weakly basic drugs are much less soluble in the pH of the small intestine, again, compromising bioavailability.

### SUMMARY

Thus, in accordance with the present disclosure, there is a pharmaceutical formulation having a weakly basic drug and an ionic polymer excipient formed as an amorphous solid dispersion. Also in this disclosure, there is provided a method of making a pharmaceutical composition comprising (a) a weakly basic drug and an ionic polymer; (b) compounding the materials of step (a) in a thermokinetic mixer for less than 300 seconds or using a hot melt extrusion process, wherein the compounding of the weakly basic drug and the ionic polymer forms an amorphous solid dispersion.

A pharmaceutical formulation of the present disclosure may include a weakly basic drug. In one embodiment, the weakly basic active pharmaceutical ingredient has a solubility of 100 μg/mL or less, such as between 1 gn/mL and 100 μg/mL inclusive, at a pH equal to or greater than 5.0. In another embodiment, the weakly basic active pharmaceutical ingredient has a solubility of 100 μg/mL or less, such as between 1 ng/mL and 100 μg/mL inclusive, when in pharmaceutically relevant neutral dissolution media, such as, FaSSIF or FeSSIF. In another embodiment, the weakly basic drug contains a primary, secondary, or tertiary amine functional group. Examples of a weakly basic drug include BI 639667, ciprofloxacin, mitoxantrone, epirubicin, daunorubicin, doxorubicin, vincristine, vinblastine, lidocaine, chlorpromazine, dibucaine, propranolol, timolol, quinidine, pilocarpine, physostigmine, dopamine, serotonin, imipramine, diphenhydramine, quinine, chloroquine, quinacrine, ritonavir, itraconazole, posaconazole, nevirapine, aprepitant, albendazole, mebendazole, amprenavir, abiraterone, saquinavir, rifabutin, anthracyclines, vinca alkaloids, lamivudine, zalcitabine, didanosine, efavirenz, zidovudine, nelfinavir, indinavir, chloroquine, azathioprine, atazanavir, amiodarone, terfenadine, tamoxifen, velpatasvir, elbasvir, and codeine, pharmaceutically acceptable salts thereof and combinations thereof.

A pharmaceutical formulation of the present disclosure may include an ionic polymer as an excipient. Examples of ionic polymer excipients include hydroxypropyl methylcellulose acetate succinate (HPMCAS), such as AFFINISOL® HPMCAS 716 G (Dow Chemical), AFFINISOL® HPMCAS 912 G (Dow Chemical), AFFINISOL® HPMCAS 126 G (Dow Chemical), AQOAT® LG (Shin-Etsu), AQOAT® MG (Shin-Etsu), and AQOAT® HG (Shin-Etsu), polyvinyl acetate phthalate, such as PHTHALAVIN® (Berwind Pharmaceutical Services), Hypromellose acetate succinate, hydroxypropyl methylcellulose phthalate, and methacrylic acid based copolymer, such as methacylic acid-co-ethyl acrylate, such as EUDRAGIT® L100-55 (Evonik, Germany), and methacylic acid-co-methyl methacrylate, such as EUDRAGIT® L100 or EUDRAGIT® S100.

A pharmaceutical formulation of the present disclosure may have particles including the amorphous solid dispersion with a specific surface area between 0.05 m²/g and 2 m²/gram, inclusive.

A pharmaceutical formulation of the present disclosure containing an amorphous solid dispersion of a weakly basic drug may dissolve less readily in the gastro-intestinal tract of a patient than a pharmaceutical formulation containing neat weakly basic drug, as evidenced by dissolution in 0.01 N HCl and FaSSIF. Additionally, a pharmaceutical formulation of the present disclosure may have a C_{max, acidic}/C_{eq, neutral} ratio that is less than or equal to 1.10, such as between 0.001 and 1.10, inclusive.

A pharmaceutical formulation of the present disclosure may be prepared using thermokinetic compounding, which is a method of compounding components until they are melt-blended. Thermokinetic compounding may be particularly useful for compounding heat-sensitive or thermolabile components. Thermokinetic compounding may provide brief processing times, low processing temperatures, high shear rates, and the ability to compound thermally incompatible materials.

The average maximum temperature in the thermokinetic chamber during thermokinetic compounding may be less than the glass transition temperature, melting point, or molten transition point, of the weakly basic drug or any other APIs present, one or all excipients, or one or all other components of the amorphous solid dispersion, or any combinations or sub-combinations of components.

Thermokinetic compounding may be performed in batches or in a semi-continuous fashion, depending on the product volume. When performed in a batch, semi-continuous, or continuous manufacturing process, each thermokinetic compounding step may occur for less than 5, 10, 15, 20, 25, 30, 35, 40, 45, 50, 55, 60, 70, 100, 120, 240, or 300 seconds, inclusive.

Without being limited by theory, thermokinetic compounding of the weakly basic drug together with the ionic polymer may provide an advantage in formulating the pharmaceutical formulations of the present disclosure such that the thermokinetic compounding process allows more intimate mixing of the weakly basic drug with the ionic polymer than is possible using some other methods of formulation. In one variation, less than 20% of the weakly basic drug thermally degrades from thermokinetic compounding.

A pharmaceutical formulation of the present disclosure may be prepared using hot melt extrusion, whereby an excipient blend is heated to a molten state and subsequently forced through an orifice where the extruded product is formed into its final shape in which it is solidified upon cooling. The blend is conveyed through various heating zones typically by a screw mechanism. The screw or screws are rotated by a variable speed motor inside a cylindrical barrel where only a small gap exists between the outside diameter of the screw and the inside diameter of the barrel. In this conformation, high shear is created at the barrel wall and between the screw flights by which the various components of the powder blend are well mixed and deaggregated.

A pharmaceutical formulation as disclosed herein resulting from hot melt extrusion may have a uniform shape and density and may not exhibit substantially changed solubility or functionality of any excipient. The weakly basic drug, ionic polymer excipient, or other components of the pharmaceutical formulation may lack substantial impurities. In one variation, less than 20% of the weakly basic drug thermally degrades from hot melt extrusion.

A pharmaceutical formulation as disclosed herein may be administered to the patient orally. The administration of this pharmaceutical formulation may be allow for a majority of the weakly basic drug to dissolve in the patient's small intestine. Additionally, the administration of this pharmaceutical formulation may allow for the minority of the weakly basic drug to dissolve in the patient's stomach. Additionally, the administration of this pharmaceutical formulation may allow for only between 0.05% and 30% of the weakly basic drug to dissolve in the patient's stomach. Additionally, the administration of this pharmaceutical formulation may allow for more of the weakly basic drug to dissolve in after passing through the stomach than in the gastric acid of the stomach. Additionally, the administration of this pharmaceutical formulation may allow for the weakly basic drug to not reach a point of crystalline drug supersaturation in the stomach and reach its point of supersaturation after leaving the stomach. Additionally, the weakly basic drug may have a max concentration in the blood plasma of the patient of greater than or equal to 1800 ng/mL, such as between 1800 ng/mL and 5,000 ng/mL or 10,000 ng/mL, inclusive. Additionally, the weakly basic drug may have a AUC₀₋₂₄ hr value of greater than or equal to 20,000 (ng×hr)/mL, such as between 20,000 (ng×hr)/mL and 50,000 (ng×hr)/mL or 100,000 (ng×hr)/mL, inclusive, in the blood plasma of the patient.

It is contemplated that any method or composition described herein can be implemented with respect to any other method or composition described herein.

It is contemplated that any embodiment discussed in this specification can be implemented with respect to any method or composition of the invention, and vice versa. Furthermore, compositions and kits of the invention can be used to achieve methods of the invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present disclosure may be further understood through reference to the attached figures in combination with the detailed description that follows.

FIG. 1 is an exemplary processing profile of thermokinetically produced amorphous solid dispersion containing BI 667 and HPMCAS-MMP.

FIG. 2 is a schematic of the post-processing for a thermokinetically produced material (KSD) after it was thermokinetically processed. The schematic shows the two mechanisms of milling/sieving that were employed and the six different species of amporhous solid dispersions that were collected.

FIG. 3 is a schematic of the post-processing for spray dry produced material (SDD) after it was processed through spray drying. The schematic shows the two different slugging forces that were used to produce two different dry granulated materials, and the unaltered spray dried material which was also collected.

FIG. 4 is a graph reporting exemplary powder X-ray diffraction patterns overlay of crystalline BI 667, BI 667:HPMCAS-MMP physical mixture, BI 667:HPMCAS-MMP produced by spray drying, amorphous BI 667, amorphous BI 667:HPMCAS-MMP produced thermokinetically, and amorphous BI 667:HPMCAS-MMP produced thermokinetically and then cyromilled (KSD CM).

FIG. 5 is a graph reporting exemplary FTIR spectra of crystalline BI 667, BI 667:HPMCAS-MMP physical mixture, HPMCAS-MPP, amorphous BI 667 produced by melt quenching, amorphous BI 667:HPMCAS-MMP produced by spray drying, amorphous BI 667:HPMCAS-MMP produced thermokinetically, and amorphous BI 667:HPMCAS-MMP produced thermokinetically and then cyromilled.

FIG. 6 is a graph reporting exemplary 1D ¹³C cross-polarization magic angle spinning (CP-MAS) spectra of BI 667:HPMCAS-MMP produced by spray drying, BI 667:HPMCAS-MMP produced thermokinetically, amorphous BI 667 produced by melt quenching, and HPMCAS-MMP.

FIG. 7 is an exemplary of the 2D ¹³C-¹H heteronuclear spectra of the BI 667:HPMCAS-MMP produced thermokinetically (blue) and BI 667:HPMCAS-MMP produced by spray drying (yellow).

FIG. 8 is an exemplary of the 2D ¹³C-¹H heteronuclear spectra of BI 667:HPMCAS-MMP produced thermokinetically (blue), HPMCAS-MPP, amorphous BI 667 produced by melt quenching (cyan), and HPMCAS-MMP (grey).

FIG. 9 is an exemplary graph reporting concentration of dissolved BI 667 versus time (dissolution profile) for the BI 667:HPMCAS-MMP physical mixture, BI 667:HPMCAS-MMP produced thermokinetically and then cyromilled, and BI 667:HPMCAS-MMP produced thermokinetically at varying particle sizes (<75 μm, 75-125 μm, 125-250 μm, 250-425 μm, 425-600 μm).

FIG. 10 is an exemplary graph reporting concentration of dissolved BI 667 versus time (dissolution profile) for the BI 667:HPMCAS-MMP physical mixture and three forms of BI 667:HPMCAS-MMP produced by spray drying (high pressure slugs, low pressure slugs, and native particles).

FIG. 11 is an exemplary graph reporting concentration of dissolved BI 667 versus time (dissolution profile) of the four partices selected for pharmacokinetic analysis in a male Beagle dog study: BI 667:HPMCAS-MMP native spray dried particles, BI 667:HPMCAS-MMP produced thermokinetically (particle sizes 75-125 μm and 425-600 μm), and BI 667:HPMCAS-MMP physical mixture.

FIG. 12 is an exemplary graph reporting concentration of dissolved BI 667 versus time in a dissolution permeation study using a Pion μFLUX™ apparatus for BI 667:HPMCAS-MMP native spray dried particles and BI 667:HPMCAS-MMP produced thermokinetically (particle sizes 75-125 μm and 425-600 μm).

FIG. 13 is an exemplary graph reporting calculated flux as a result of BI 667 concentrations in the acceptor compartment during different time portions of the dissolution premation study of BI 667:HPMCAS-MMP native spray dried particles and BI 667:HPMCAS-MMP produced thermokinetically (particle sizes 75-125 μm and 425-600 μm).

FIG. 14 is an exemplary graph reporting BI 667 concentration versus time profiles following oral administration to male beagle dogs of BI 667:HPMCAS-MMP native spray dried particles, BI 667:HPMCAS-MMP produced thermokinetically (sizes 75-125 μm and 425-600 μm), and BI 667:HPMCAS-MMP physical mixture.

FIG. 15A is an exemplary scanning electron microscopy image of the surface structure of BI 667:HPMCAS-MMP produced thermokinetically (particle size 75-125 μm).

FIG. 15B is an exemplary scanning electron microscopy image of the surface structure of BI 667:HPMCAS-MMP native spray dried particles.

FIG. 15C is an exemplary scanning electron microscopy image of the surface structure of BI 667:HPMCAS-MMP (produced thermokinetically and then cyromilled).

FIG. 15D is an exemplary scanning electron microscopy image of the surface structure of the BI 667:HPMCAS-MMP physical mixture imaged.

FIG. 16 is an exemplary graph reporting BI 667 concentration versus time profiles of BI 667:HPMCAS-MMP native spray dried particles, BI 667:HPMCAS-MMP produced thermokinetically (75-125 μm), and amorphous BI 667 produced by melt quenching; prepared at 1.33 mg/mL in 0.5% methylcellulose and 0.1% Tween 20 suspension in water; tested by adding 20 mL of the suspension to the PION at 150 RPM.

FIG. 17A is an exemplary graph reporting concentrion of dissolved BI 667 versus time (dissolution profile) for the BI667:HPMCAS-HMP, BI 667:HPMCAS-LMP and BI 667:L100-55 produced thermokinetically and spray dried. FIG. 17B and FIG. 17C separate the data in FIG. 17A into two different figures but represent the same data as in FIG. 17A.

### DETAILED DESCRIPTION

The present disclosure relates to pharmaceutical formulations containing a weakly basic drug and an ionic polymer and methods of forming and administering such pharmaceutical formulations.

### A. PHARMACEUTICAL FORMULATION

A pharmaceutical formulation of the present disclosure may include a weakly basic drug as an active pharmaceutical ingredient (API). In one embodiment, the weakly basic drugs of the compositions of the present invention may refer to a compound:

a) having a pKa of 14 or less, such as a pKa of between 1 and 14, inclusive;

b) having a solubility of 100 μg/mL or less, at a pH equal to or greater than 5.0, such as between 1 ng/mL and 100 μg/mL, inclusive, at a pH equal to or greater than 5.0;

c) at least one basic nitrogen atom; or

d) any combinations of features a), b), and c).

In another embodiment, the weakly basic drug may contain a primary, secondary, or tertiary amine functional group. Examples of a weakly basic drugs include BI 639667, ciprofloxacin, mitoxantrone, epirubicin, daunorubicin, doxorubicin, vincristine, vinblastine, lidocaine, chlorpromazine, dibucaine, propranolol, timolol, quinidine, pilocarpine, physostigmine, dopamine, serotonin, imipramine, diphenhydramine, quinine, chloroquine, quinacrine, ritonavir, itraconazole, posaconazole, nevirapine, aprepitant, albendazole, mebendazole, amprenavir, abiraterone, saquinavir, rifabutin, anthracyclines, vinca alkaloids, lamivudine, zalcitabine, didanosine, efavirenz, zidovudine, nelfinavir, indinavir, chloroquine, azathioprine, atazanavir, amiodarone, terfenadine, tamoxifen, velpatasvir, elbasvir, and codeine, pharmaceutically acceptable salts thereof, and combinations thereof.

A pharmaceutical formulation of the present disclosure may include an ionic polymer as an excipient. Ionic polymer excipients include hydroxy propyl methyl cellulose acetate succinate, such as AFFINISOL® HPMCAS 716 G (Dow Chemical), AFFINISOL® HPMCAS 912 G (Dow Chemical), AFFINISOL® HPMCAS 126 G (Dow Chemical), AQOAT® LG (Shin-Etsu), AQOAT® MG (Shin-Etsu), and AQOAT® HG (Shin-Etsu) polyvinyl acetate phthalate, such as PHTHALAVIN® (Berwind Pharmaceutical Services), Hypromellose acetate succinate, hydroxypropyl methylcellulose phthalate, and methacrylic acid based copolymers, for example either, methacylic acid-co-ethyl acrylate, such as EUDRAGIT® L100-55 (Evonik, Germany), or methacylic acid-co-methyl methacrylate, such as EUDRAGIT® L100 or EUDRAGIT® S100.

The ionic polymer excipient may be used alone, or a pharmaceutical formulation of the present disclosure may include a combination of ionic polymer excipients.

A pharmaceutical formulation of the present disclosure may be in the form of an amorphous solid dispersion of the weakly basic drug and the ionic polymer excipient. The amorphous nature of the solid dispersion may be confirmed using X-ray diffraction (XRD), which may not exhibit strong peaks characteristic of a crystalline material.

A pharmaceutical formulation of the present disclosure may have a weight ratio of weakly basic drug to ionic polymer of between 1:0.25 to 1:50, inclusive.

A pharmaceutical formulation of the present disclosure may be formed by any suitable method for making amorphous solid dispersions, such as thermokinetic compounding or hot-melt extrusion. Thermokinetic compounding may be particularly useful for excipients and weakly basic drugs that experience degradation in hot melt extrusion. Thermokinetic compounding followed by milling the resultant thermokinetically compounded product may be particularly useful in making amorphous solid dispersion particles with a surface area under 2 m²/g, inclusive.

A pharmaceutical formulation of the present disclosure may have a specific surface area between 0.05 m²/g and 2 m²/g, inclusive.

A pharmaceutical formulation of the present disclosure containing an amorphous solid dispersion of a weakly basic drug may dissolve less readily in the gastrointestinal tract of a patient than a pharmaceutical formulation containing neat crystalline weakly basic drug, as evidenced by dissolution in 0.01 N HCl and FaSSIF. Additionally, a pharmaceutical formulation of the present disclosure may have a C_{max, acidic}/C_{eq, neutral} ratio of a weakly basic drug that is less than or eq 1.10, such as 0.001 and 1.10, inclusive. A C_{max, acidic}/C_{eq, neutral} ratio may be a ratio found through a non-sink, pH dissolution test that includes comparing the maximum concentration over the first 30 minutes of adding 120 mg of the pharmaceutical formulation to 90 mL of 0.01 N HCl (C_{max, acidic}) to the maximum concentration over the next 330 minutes after adding 60 mL of FaSSIF (2.24 g/L SIF in 0.1 M sodium phosphate buffer, pH 6.8) to the solution at the 30 minute mark.

A pharmaceutical formulation of the present disclosure may be for oral administration and may be further processed, with or without further compounding, to facilitate oral administration.

A pharmaceutical formulation of the present disclosure may be further processed into a solid dosage form suitable for oral administration, such as a tablet or capsule.

### B. METHODS OF FORMULATING A PHARMACEUTICAL FORMULATION

A pharmaceutical formulation of the present disclosure may be prepared using thermokinetic compounding, which is a method of compounding components until they are melt-blended. Thermokinetic compounding may be particularly useful for compounding heat-sensitive or thermolabile components. Thermokinetic compounding may provide brief processing times, low processing temperatures, high shear rates, and the ability to compound thermally incompatible materials.

Thermokinetic compounding may be carried out in a thermokinetic chamber using one or multiple speeds during a single, compounding operation on a batch of components to form a pharmaceutical formulation of the present disclosure.

A thermokinetic chamber includes a chamber having an inside surface and a shaft extending into or through the chamber. Extensions extend from the shaft into the chamber and may extend to near the inside surface of the chamber. The extensions are often rectangular in cross-section, such as in the shape of blades, and have facial portions. During thermokinetic compounding, the shaft is rotated causing the components being compounded, such as particles of the components being compounded, to impinge upon the inside surface of the chamber and upon facial portions of the extensions. The shear of this impingement causes comminution, frictional heating, or both of the components and translates the rotational shaft energy into heating energy. Any heating energy generated during thermokinetic compounding is evolved from the mechanical energy input. Thermokinetic compounding is carried out without an external heat source. The thermokinetic chamber and components to be compounded are not pre-heated prior to commencement of thermokinetic compounding.

The thermokinetic chamber may include a temperature sensor to measure the temperature of the components or otherwise within the thermokinetic chamber.

During thermokinetic compounding, the average temperature of the thermokinetic chamber may increase to a pre-defined final temperature over the duration of the thermokinetic compounding to achieve thermokinetic compounding of the weakly basic drug and the ionic polymer excipient, and any other components of a pharmaceutical formulation of the present disclosure, such as an additional API, an additional excipient, or both. The pre-defined final temperature may be such that degradation of the weakly basic drug, ionic polymer excipient, or other components is avoided or minimized. Similarly, the one or multiple speeds of use during thermokinetic compounding may be such that thermal degradation of the weakly basic drug, ionic polymer excipient, or other components is avoided or minimized. As a result, the weakly basic drug, ionic polymer excipient, or other components of the amorphous solid dispersion may lack substantial impurities. In one variation, less than 20% of the weakly basic drug thermally degrades from thermokinetic compounding. In particular, between 0.0001% and 20%, inclusive, of the weakly basic drug thermally degrades from thermokinetic compounding.

The average maximum temperature in the thermokinetic chamber during thermokinetic compounding may be less than the glass transition temperature, melting point, or molten transition point, of the weakly basic drug or any other APIs present, one or all excipients, or one or all other components of the amorphous solid dispersion, or any combinations or sub-combinations of components.

Pressure, duration of thermokinetic compounding, and other environmental conditions such as pH, moisture, buffers, ionic strength of the components being mixed, and exposure to gasses, such as oxygen, may also be such that degradation of weakly basic drug or any other APIs present, one or all excipients, or one or all other components is avoided or minimized.

Thermokinetic compounding may be performed in batches or in a semi-continuous fashion, depending on the product volume. When performed in a batch, semi-continuous, or continuous manufacturing process, each thermokinetic compounding step may occur for less than 5, 10, 15, 20, 25, 30, 35, 40, 45, 50, 55, 60, 70, 100, 120, 240, or 300 seconds, inclusive, or for an interval between any of these time points, inclusive, or for an interval between 1 second any any of these time points, inclusive.

Variations of thermokinetic compounding may be used depending on the amorphous solid dispersion and its components. For example, the thermokinetic chamber may be operated at a first speed to achieve a first process parameter, then operated at a second speed in the same thermokinetic compounding process to achieve a final process parameter. In other examples, the thermokinetic chamber may be operated at more than two speeds, or at only two speeds, but in more than two-time internals, such as at a first speed, then at a second speed, then again at the first speed.

The weakly basic drug component may be in a crystalline or semi-crystalline form prior to thermokinetic compounding.

In another variation, a weakly basic drug or other API particle size is reduced prior to thermokinetic compounding. This may be accomplished by milling, for example dry milling the crystalline form of the weakly basic drug or other API to a small particle size prior to thermokinetic compounding, wet milling the crystalline form of the weakly basic drug or other API with a pharmaceutically acceptable solvent to reduce the particle size prior to thermokinetic compounding, or melt milling the crystalline form of the weakly basic drug or other API with at least one excipient having limited miscibility with the crystalline form of the weakly basic drug or other API to reduce the particle size prior to thermokinetic compounding.

Another variation includes milling the crystalline form of the weakly basic drug or other API in the presence of an excipient to create an ordered mixture where the weakly basic drug or other API particles adhere to the surface of excipient particles, excipient particles adhere to the surface of API particles, or both.

Without being limited by theory, thermokinetic compounding of the weakly basic drug together with the ionic polymer may provide an advantage in formulating the pharmaceutical formulations of the present disclosure such that the thermokinetic compounding process allows more intimate mixing of the weakly basic drug with the ionic polymer than is possible using some other methods of formulation.

The pharmaceutical formulation of the present disclosure may be formulated without a solvent. For example, the pharmaceutical formulation of the present disclosure may be prepared using thermokinetic compounding without a solvent. Accordingly, a pharmaceutical formulation of the present disclosure prepared by thermokinetic compounding may have no solvent in the pharmaceutical formulation or a tablet thereof and may have no impurities comprising the solvent in the pharmaceutical formulation or a tablet thereof.

A pharmaceutical formulation of the present disclosure may be prepared using hot melt extrusion, whereby an excipient blend is heated to a molten state and subsequently forced through an orifice where the extruded product is formed into its final shape in which it is solidified upon cooling. The blend is conveyed through various heating zones typically by a screw mechanism. The screw or screws are rotated by a variable speed motor inside a cylindrical barrel where only small gap exists between the outside diameter of the screw and the inside diameter of the barrel. In this conformation, high shear is created at the barrel wall and between the screw flights by which the various components of the powder blend are well mixed and deaggregated.

The hot-melt extrusion equipment is typically a single or twin-screw apparatus but can be composed of more than two screw elements. A typical hot-melt extrusion apparatus contains a mixing/conveying zone, a heating/melting zone, and a pumping zone in succession up to the orifice. In the mixing/conveying zone, the powder blends are mixed and aggregates are reduced to primary particles by the shear force between the screw elements and the barrel. In the heating/melting zone, the temperature is at or above the melting point or glass transition temperature of the thermal binder or binders in the blend such that the conveying solids become molten as they pass through the zone. A thermal binder in this context describes an inert excipient, typically a polymer, that is solid at ambient temperature, but becomes molten or semi-liquid when exposed to elevated heat or pressure. The thermal binder acts as the matrix in which the weakly basic drug and other APIs are dispersed, or the adhesive with which they are bound such that a continuous composite is formed at the outlet orifice. Once in a molten state, the homogenized blend is pumped to the orifice through another heating zone that maintains the molten state of the blend. At the orifice, the molten blend may be formed into strands, cylinders or films. The extrudate that exits is then solidified typically by an air-cooling process. Once solidified, the extrudate may then be further processed to form pellets, spheres, fine powder, tablets, and the like.

A pharmaceutical formulation as disclosed herein resulting from hot melt extrusion may have a uniform shape and density and may not exhibit substantially changed solubility or functionality of any excipient. The weakly basic drug, ionic polymer excipient, or other components of the pharmaceutical formulation may lack substantial impurities. In one variation, less than 20% of the weakly basic drug thermally degrades from hot melt extrusion. In particular, between 0.0001% and 20%, inclusive, of the weakly basic drug thermally degrades from hot melt extrusion.

In some examples, the pharmaceutical formulation may be tableted, then coated with a composition containing another API.

### C. METHODS OF ADMINISTERING A PHARMACEUTICAL FORMULATION

A pharmaceutical formulation of the present disclosure may be administered to a patient orally.

When a pharmaceutical formulation of the present disclosure is administered to a patient orally, at least 50%, inclusive, of the weakly basic drug may dissolve in the small intestine of the patient, as opposed to in other organs of the gastrointestinal tract. In the present disclosure, the path a pharmaceutical formulation takes through a patient's body may include entering the stomach, and after a period of time in the stomach, entering the small intestine. The weakly basic drug may reach saturation concentration in the small intestine contents of the patient. The weakly basic drug may have a max concentration in the blood plasma of the patient of greater than or equal to 1800 ng/mL, such as between 1800 ng/mL and 5,000 ng/mL or 10,000 ng/mL, inclusive. Additionally, the weakly basic drug may have a AUC₀₋₂₄ hr value of greater than or equal to 20,000 (ng×hr)/mL, such as between 20,000 (ng×hr)/mL and 50,000 (ng×hr)/mL or 100,000 (ng×hr)/mL, inclusive, in the blood plasma of the patient.

When a pharmaceutical formulation of the present disclosure is administered to a patient orally, between 0.05% and 30%, inclusive, of the weakly basic drug may dissolve in the patient's stomach. The weakly basic drug may not reach saturation concentration in the stomach contents of the patient.

A pharmaceutical formulation of the present disclosure may be particularly useful when the patient has experienced a sub-optional response to or been resistant to formulations containing a crystalline form of the weakly basic drug or an amorphous solid dispersion form of the weakly basic drug where the specific surface area of the particles is above 2.0 m²/g.

A pharmaceutical formulation of the present disclosure may be useful for the administration of weakly basic drugs that exhibit a higher absorption rate in the small intestine than in other organs of the gastro-intestinal tract.

In general, a pharmaceutical formulation of the present disclosure may be used to administer any amount of weakly basic drug to a patient on any schedule.

### D. EXAMPLES

The present examples are provided for illustrative purposes only. They are not intended to and should not be interpreted to encompass the full breadth of the disclosure.

Various compositions and instruments are identified by trade name in this application. All such trade names refer to the relevant composition or instrument as it existed as of the earliest filing date of this application, or the last date a product was sold commercially under such trade name, whichever is later. One of ordinary skill in the art will appreciate that variant compositions and instruments sold under the trade name at different times will typically also be suitable for the same uses.

### Example 1: Solid Dispersions of a Weakly Basic Drug and Ionic Polymers

Solid dispersions, with a 1:2 drug-polymer ratio, some of which were amorphous solid dispersions were prepared via thermokinetic compounding using a lab-scale thermokinetic compounder (DisperSol Technologies LLC, Georgetown, Tex.). These solid dispersions contained a weakly basic drug, Boehringer Ingelheim research compound BI 639667 (BI 667), and a ionic polymer, HPMCAS-MMP, at a 1:2 drug-polymer ratio.

To form these solid dispersion, 15 g of a physical mixture of BI 667 and HPMCAS-MMP was added to a plastic container and mixed in a TURBULA® T2F Shaker-Mixer (Glen Mills Inc., Clifton, N.J.) for 5 minutes prior to charging into the compounder chamber the the thermokinetic compounder.

Inside the compounder chamber, a shaft with protruding novel mixing elements was rotated at a speed of 500 rpm for 5 seconds followed by a speed of 4,500 rpm until the desired set point ejection temperature was achieved. No external heat input was added to the system, though frictional and shear forces cause the sample's temperature to rise. The chamber's temperature was monitored using a real-time infrared probe. When the molten material reached a temperature of 160° C., the mass was rapidly ejected, collected, and pressed between two aluminum plates to rapidly quench the sample and arrest any further reaction. The quenched material was labeled as a thermokinetic amorphous solid dispersion (KSD).

The rotation speed of the novel mixing elements and set point ejection temperature were optimized after extensive preliminary batch processing at various rotation speeds and ejection temperatures. The overall goal of preliminary processing was to optimize modifiable parameters (such as rotation speed of novel mixing elements and the set point ejection temperature) to prevent degradation while generating KSD samples. To ensure that a ‘steady-state’ of thermokinetic processing was achieved before collecting samples, two batches of KSDs were produced before a third was generated and kept for post-thermokinetic processing such as milling.

Following thermokinetic processing, the quenched KSD sample was broken into fragments and the particle size of the fragments were reduced using a mortar and pestle. The particles produced were then sieved through a series of Advantech sieves using an Advantech Sonic Sifter (Advantech Manufacturing, Inc., New Berlin, Wis.). Sieves with mesh sizes of 600, 425, 250, 125, and 75 μm were used to isolate multiple particle size species of KSD particles. The particles of KSD material were typically not rounded and had a structure dependent on the processing parameters as well as the drug and polymer selected.

The particles generated from the mortar and pestle step were loaded on top of the 600 μm sieve and sifted to pass through each sieve (i.e., 600→425→250→125→75) until they were retained on a mesh. Species of KSD particles were collected for further analysis based on the mesh at which they were retained. For example, KSD particles that passed through the 125 μm mesh but were retained on the 75 μm mesh were labeled ‘KSD 75-125’: KSD<75, KSD 75-125, KSD 125-250, KSD 250-425, and KSD 425-600 samples were all created. All sieved KSD species were stored at room temperature in a vacuum sealed desiccator for further analysis. During solid state characterization, when KSD species is not specified, KSD 75-125 was utilized.

A second milling process, cryomilling, was utilized to help understand the dissolution behavior of KSD particles. For this process, an aliquot of the fragments produced following thermokinetic processing were loaded into a cryomill tube with impactor. The tube was sealed and loaded into a SPEX 6870 Freezer/Mill (SPEX SamplePrep, Metuchen, N.J.) and immersed in a liquid nitrogen bath. Following a pre-cool time of 1 min, the KSD sample was impacted by oscillation at 10 cps for a 2-min duration followed by a 1 min cooling time, and the oscillation/cooling steps were repeated for a total of 10 iterations. The resulting material was immediately transferred to a desiccator with an active vacuum until the material reached room temperature. After the cryomilled material reached room temperature, the material was collected and labeled KSD CM (cryomilled). The KSD CM particles were stored at room temperature in a vacuum sealed desiccator for further analysis.

Compounds for comparison were also created, in the form of a spray dried dispersion (SDD) and amorphous BI 677.

Prior to spray drying, 15 g of BI 667:HPMCAS-MMP (1:2) was solubilized in acetone:deionized water (9:1) at 3.33% w/v and stirred overnight to ensure homogeneity of the drug-polymer-solvent system. Spray drying was conducted on a Büchi Mini Spray Dryer B-290 with Inert Loop B-295 (Flawil, Switzerland) and a 1.50 mm spray nozzle. Nitrogen gas was used to create a low oxygen environment and for atomization of the spray solution. The feed solution flow rate was kept constant at 4.5 mL/min by a Masterflex® L/S® Cole-Parmer peristaltic pump (Cole-Parmer, Vernon Hills, Ill.). The inlet temperature was set to 78° C. to produce an outlet temperature between 57-58° C., and the inert loop was set to 1° C. The atomizing gas flow rate was held constant at 9 L/min, and the aspirator flow was set at 35 m³/hr. The resulting spray dried product was collected and dried in a vacuum oven at 40° C. for 24 hours. After the secondary drying step, the spray dried material was stored at room temperature in a vacuum sealed desiccator. The collected material was labeled spray dried dispersion (SDD).

Following spray drying, a portion of the collected SDD was compressed into slugs and subsequently milled through a series of sieves in order to densify the particles and impact release properties in dissolution. A GlobePharma Manual Tablet Compression Machine, MTCM-I, (GlobePharma Inc., New Brunswick, N.J.) equipped with a 25-mm die was used to form slugs of SDD. Two different pressures were applied with the tablet press in order to generate low pressure (LP) and high pressure (HP) slugs of SDD. For LP slugs, 2,500 psi of pressure was applied to approximately 500 mg of SDD material for 5 s. For HP slugs, 7,500 psi of pressure was applied to approximately 500 mg of SDD material for 10 s. The LP and HP slugs were milled and sieved separately through a series of Advantech sieves, and the final products were collected after the material passed through a 125 μm sieve. The resulting granules were labeled SDD LP and SDD HP for SDD material that underwent low and high pressure during the compression step, respectively. The SDD LP and SDD HP granules were then stored at room temperature in a vacuum sealed desiccator.

To obtain an amorphous BI 667 reference material necessary for characterization, a melt quench method was employed. Crystalline BI 667 was heated to 220° C. (˜15° C. above its melting point) and held isothermal for 5 min in a Breville Smart Oven® Pro (Breville USA, Torrance, Calif.). After 5 min, the melted BI 667 was removed from the oven and rapidly quenched with liquid nitrogen until the sample was completely cooled. The resulting material was collected, labeled Amorphous BI 667, and stored at room temperature in a vacuum sealed desiccator.

### Example 2: Thermostability Testing

Prior to thermokinetic processing, thermal stability of BI 667 was assessed using thermogravimetric analysis (TGA). BI 667 was considered stable (<1% mass loss on TGA) at relevant processing temperatures (<250° C.). HPMCAS-MMP begins to degrade by 220° C., and a BI 667:HPMCAS-MMP (1:2) physical mixture began degrading around 240° C. (data not shown). However, preliminary processing of BI 667:HPMCAS-MMP (1:2) blends indicated processing near 180° C. in the compounding chamber led to degradation. Thus, a max processing temperature (and therefore ejection set point temperature) of the mixture was set at 160° C.

TGA was performed on a TGA/DSC 1 (Mettler Toledo, Schwerzenbach, Switzerland). The temperature ramp utilized in this study was performed from 25 to 300° C. at a rate of 5° C./min with a nitrogen purge of 50 mL/min. Data were analyzed using STARE System.

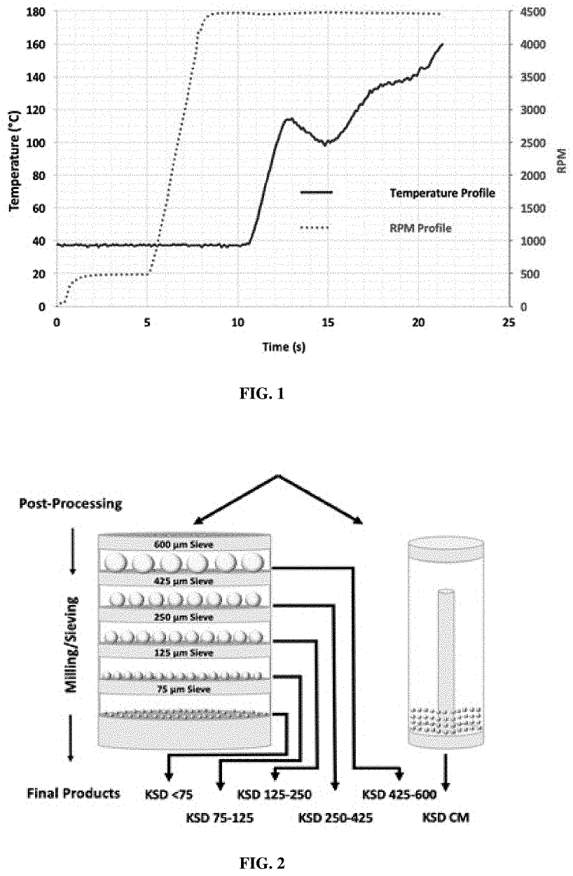

Thermokinetically processed samples were produced by a two-stage mixing profile as shown in FIG. 1. The contents inside the compounding chamber were mixed at 500 rpm for 5 s before ramping the mixing rate to 4,500 rpm. The mixing rate was held constant at 4,500 rpm until the set point ejection temperature (160° C.) was achieved. The temperature profile illustrates the contents inside the chamber were processed in <22 s, and the time at elevated temperatures (>40° C.) was <11 s. The processing profile is representative of a standard run with the BI 667:HPMCAS-MMP (1:2) mixture when 15 g of material is loaded into the compounding chamber.

After the set point ejection temperature was achieved, the sample was rapidly ejected from the compounding chamber and immediately quenched between two aluminum plates to arrest any further processing. The ejected sample was labeled thermokinetic solid dispersion (KSD). The KSD sample was then fractured into large fragments and milled and sieved as illustrated in FIG. 2. A majority of the processed KSD sample was milled and passed through a series of sieves, as shown in FIG. 2. Five sieves were selected with decreasing mesh sizes to produce different species of KSD particles. Aliquots of KSD material were collected on top of the 425, 250, 125, and 75 μm sieves, and KSD material that passed through the 75 μm sieve was also collected. KSD samples were labeled according to the mesh size on which they were retained followed by the mesh they were last passed through. Five different species were collected and further analyzed: KSD<75, KSD 75-125, KSD 125-250, KSD 250-425, and KSD 425-600.

A small portion of processed KSD material that was not milled and sieved as described above was cryomilled as previously described. The material was collected without further sieving and labeled KSD CM.

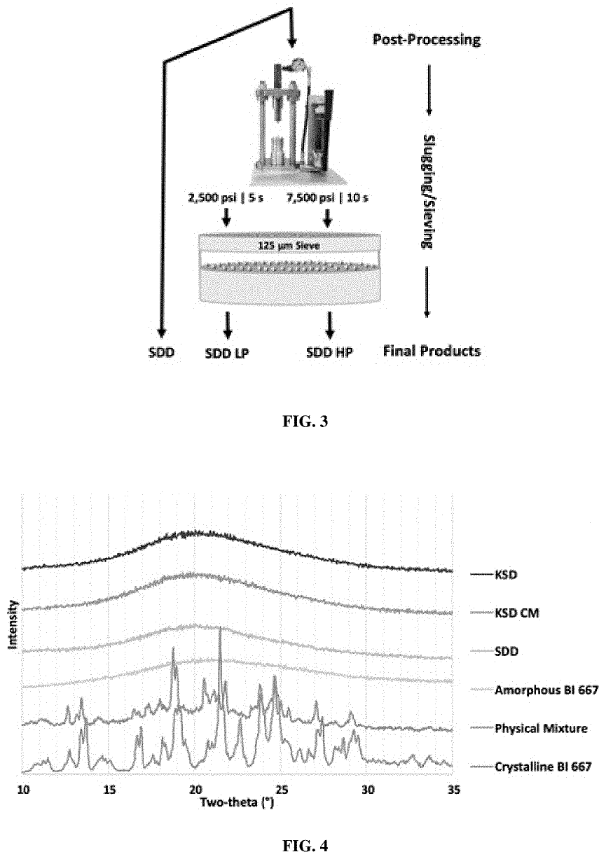

Spray drying was conducted as described above, and the SDD material collected after the secondary drying step was processed as illustrated in FIG. 3. The unaltered SDD material was collected immediately after secondary drying (vacuum oven drying) for further analysis. However, a portion of the SDD was dry granulated using two different slugging pressures to impart different stresses on the SDD particles, and ultimately the material was sieved through a 125 μm sieve. The SDD LP (low pressure) slugs were friable and easily fractured and passed through the 125 μm sieve after little manipulation with a pestle. The SDD HP (high pressure) slugs, which were subjected to 3× higher pressure and 2× longer retention time in the tablet press, formed slugs that were notably more rigid and were less prone to breaking. After impacting the slugs with a pestle, the SDD HP slugs passed through a 125 μm sieve and collected. Both the SDD LP and SDD HP materials were collected and further analyzed.

### Example 3: Solid State Characterization and Molecular Interactions

Powder X-ray diffraction (PXRD) experiments were conducted on a Rigaku MiniFlex600 II (Rigaku Americas, The Woodlands, Tex.) instrument equipped with a Cu-Kα radiation source generated at 40 kV and 15 mA. The two-theta angle range, step size, and scan speed were set to 10-35°, 0.02°, and 2°/min, respectively. Aluminum holders with a glass sample holder adapter were set on a rotating stage while the diffractometer scanned over the powder samples. The powder samples were prepared as described previously. To obtain PXRD patterns, the raw data were compiled using MDI JADE 9 software (Materials Data Inc., Livermore, Calif.) and exported to Microsoft Excel (Microsoft Corporation, Redmond, Wash.) for plotting.

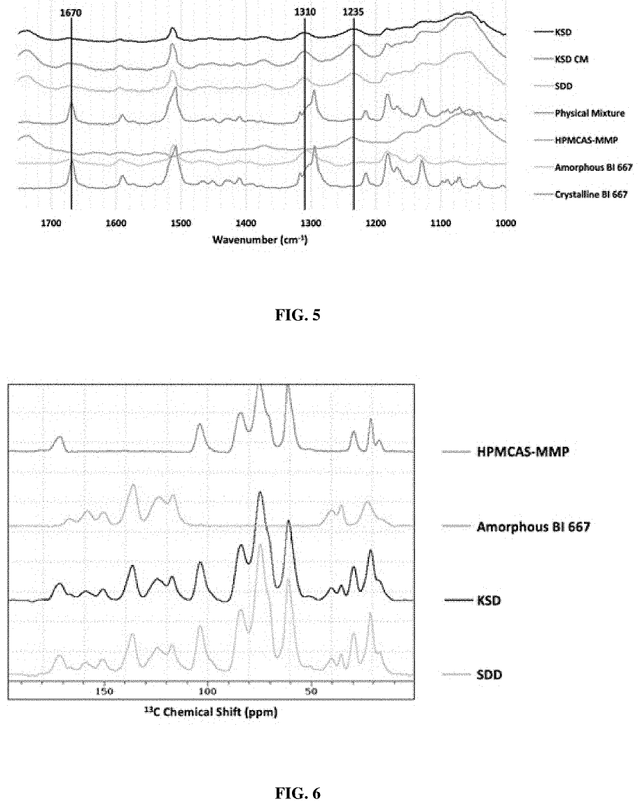

KSD, KSD CM, and SDD were analyzed by PXRD and were compared with neat BI 667, a BI 667:HPMCAS-MMP (1:2) physical mixture, and an amorphous BI 667 reference formed by melt quenching BI 667, and an overlay of the scans are shown in FIG. 4. As seen in the crystalline BI 667 and physical mixture samples, BI 667 exhibits major Bragg's peaks at 18.9, 21.5, 23.8, and 24.7 two-theta degrees. No Bragg's peaks related to BI 667, or of any kind, were detected by PXRD for all processed samples analyzed.

Thermal analysis was conducted by modulated differential scanning calorimetry (mDSC) with a Q20 Differential Scanning calorimeter (TA Instruments, New Castle, Del.). Sample was prepared in a standard aluminum pan and lid with pinhole and was crimped and sealed with a Tzero press (TA Instruments). Approximately 3-6 mg of sample was prepared and accurately weighed with a Sartorius 3.6P microbalance (Gottingen, Germany). To determine the presence/absence of a melting point (Tₘ), and for initial thermal analysis of neat BI 667, a standard mDSC method was conducted. Following sample equilibration at 35° C. for 5 min, the temperature ramped at 3° C./min from 35 to 300° C. with a modulation of 0.3° C. every 50 s.

For glass transition temperature (T_{g}) determination, samples were heated 20° C./min from 35-250° C. and held isothermal at 250° C. for 5 min. Samples were then cooled at 20° C./min to 35° C. and held isothermal for 5 min. Finally, samples were heated at 3° C./min from 35-250° C. with a modulation of 0.3° C. every 50 s. Nitrogen was used as the sample purge gas at 50 mL/min throughout all studies. All samples were analyzed using Universal Analysis 2000 software (TA Instruments).

Thermal analysis of neat BI 667 using mDSC indicated BI 667 had a Tₘ and T_{g} of 206 and 97° C., respectively. KSD, KSD CM, and SDD samples were analyzed by mDSC (data not shown) to evaluate for the presence of a single T_{g}. Single T_{g} values of 94.3° C., 95.1° C., and 94.8° C. were recorded of KSD, KSD CM, and SDD samples, respectively.

Molecular interactions among BI 667 and HPMCAS-MMP were evaluated with attenuated total reflectance (ATR)-Fourier-transform infrared spectroscopy (FTIR). Spectra were collected on a Nicolet™ iS™ 50 spectrometer (Thermo Scientific, Waltham, Mass.). A sufficient amount of sample to cover the crystal area was place on a germanium crystal, and constant torque was applied with the built-in pressure tower to obtain uniform contact between the sample and the crystal. A total of 64 scans were taken with 4 cm⁻¹ resolution from 700-4000 cm⁻¹ at room temperature. The normalized spectra were analyzed with OMNIC™ software.

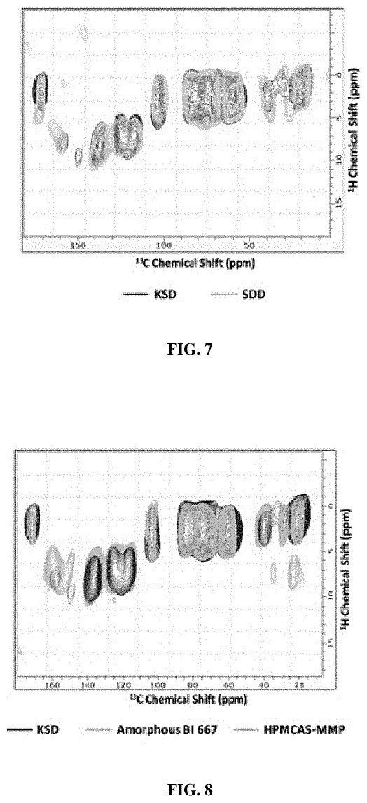

To observe potential molecular interaction differences between BI 667 and HPMCAS-MMP from the different processing methods, FTIR was employed. FIG. 5 shows a region of interest (1000-1750 cm⁻¹) from the FTIR scan, where samples containing amorphous BI 667 exhibit broad peaks at wavenumbers 1235, 1310, and 1670 cm⁻¹. KSD, KSD CM, and SDD samples all exhibit nearly identical FTIR spectra from 700-4000 cm⁻¹ (full spectra not shown).

ssNMR experiments were carried out using a 500 MHz Bruker Avance III spectrometer (Bruker Corporation, Billerica, Mass.) in the Pharmaceutical NMR lab of Preclinical Development at Merck Research Laboratories (MRLs, Merck & Co., Inc., West Point, Pa.). One-dimensional (1D) and two-dimensional (2D) spectra for ¹H, and ¹³C were obtained at a magic angle spinning (MAS) of 12 kHz with a Bruker 4 mm H/F/X MAS probe. All spectra were acquired at 298 K and processed in Bruker Topspin 3.5 software. 2D heteronuclear dipolar correlation (HETCOR) experiments between ¹H and ¹³C were obtained with a contact time of 2 ms to obtain long-range intermolecular correlation peaks, which were used to understand potential interaction differences between BI 667 and HPMCAS in the KSD and SDD samples.

The ¹H spin-lattice relaxation time in the laboratory frame, T₁, and spin-lattice relaxation time in the rotating frame, T_{1ρ}, values were measured using ¹H-¹³C cross polarization (CP) based experiments through ¹³C observation. The CP MAS and relaxation experiments were conducted with a linearly ramped power level of 80-100 kHz during a 2 ms CP contact time on the ¹H channel for enhancing polarization transfer efficiency. A high power SPINAL64 proton decoupling was utilized during the acquisition time at a field strength of 80 kHz. All data were acquired at a MAS frequency of 12 kHz at ambient temperature. The determined ¹H T₁ and T_{1ρ} relaxation values can be utilized to evaluate drug-polymer heterogeneity and provide estimations of the diffusive path length. An estimation of the upper limit to the domain size were obtained from the relaxation time, t, by the following equation:

L=\sqrt{6Dt}

Where L is magnetization diffusion across a length and describes the domain size. D is the spin diffusion coefficient of organic polymers. A constant of 8.0×10⁻¹² cm²/s is often utilized for a rigid system. The difference or similarity of relaxation values between BI 667 and HPMCAS then can be determined for evaluating drug-polymer miscibility in the KSD and SDD samples.

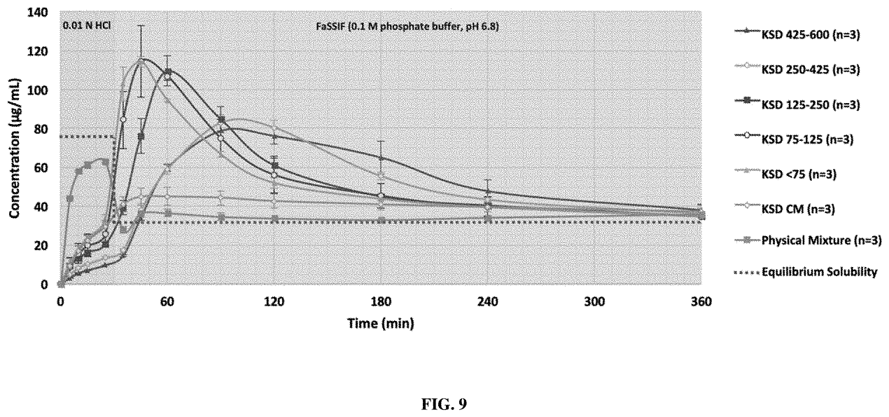

1D ¹³C cross-polarization magic angle spinning (CP-MAS) spectra of the SDD, KSD, amorphous BI 667, and HPMCAS-MMP were acquired and are shown in FIG. 6. Amorphous BI 667 exhibits broad peaks due to disordered molecular orientations, which is consistent with previously reported CP-MAS spectra of ASDs The spectral features of the KSD and SDD samples were identical and consistent with spectra of the amorphous BI 667 between 110-160 ppm.

Utilizing the 1D ¹³CP-MAS NMR spectra, the individual ¹H spin-lattice relaxation behaviors in the laboratory (T₁) and rotating (T_{1ρ}) frames were measured. The relaxation data obtained are shown in Table 1. The three following categories were assessed to determine molecular miscibility of the BI 667:HPMCAS-MMP system [45, 46]: (i) Miscible, both T₁ and T_{1ρ} values are consistent for BI 667 and HPMCAS-MMP; (ii) Partly miscible, the T₁ values for BI 667 and HPMCAS-MMP will be the same, while the T_{1ρ} values will differ; (iii) Immiscible, both T₁ and T_{1ρ} values of BI 667 and HPMCAS-MMP will differ. From the relaxation data, the miscibility of the processed ASDs were evaluated, and both KSD and SDD systems are considered molecularly miscible at the T₁ and T_{1ρ} domains.

TABLE 1
Miscibility of BI 667 and HPMCAS-MMP in KSD and SDD samples
evaluated from ¹H spin-lattice relaxation measurements

| | | T₁ | ΔT₁ | Domain | T₁ₚ | ΔT₃ₚ | Domain | |
|---|---|---|---|---|---|---|---|---|
| | | (s) | (s) | Size (nm) | (ms) | (ms) | Size (nm) | Miscibility |
| KSD | BI 667 | 5.35 ± 0.20 | 0.05 | 160 | 20.72 ± 2.26 | 1.94 | 32 | Miscible |
| | HPMCAS-MMP | 5.40 ± 0.06 | | 161 | 22.66 ± 0.46 | | 33 | |
| SDD | BI 667 | 4.92 ± 0.26 | 0.23 | 154 | 19.18 ± 1.48 | 1.10 | 30 | Miscible |
| | HPMCAS-MMP | 5.15 ± 0.41 | | 157 | 20.28 ± 0.93 | | 31 | |

2D ¹³C-¹H heteronuclear correlation (HETCOR) experiments were employed to further probe potential molecular differences between KSD and SDD samples in a 2D manner. The 2D ¹³C-¹H HETCOR spectra are shown in FIG. 7 and FIG. 8. In FIG. 7, the proton and carbon shifts associated with the KSD and SDD ASDs are nearly identical, and disparities between the spectra are likely noise and/or variance in instrument data acquisition. FIG. 8 illustrates proton and carbon shifts of the KSD sample compared with an amorphous BI 667 reference and the HPMCAS-MMP polymer. From the figure, it is apparent that a region of amorphous BI 667 (¹³C 15-20 ppm, ¹H 5-10 ppm) disappears in the KSD sample (which is also true of the SDD, though the spectra are not shown). Otherwise, the spectra between the KSD and BI 667 are nearly identical, and the spectra of the KSD and HPMCAS-MMP are in agreement.

### Example 4: Dissolution Testing of BI 667 Pharmaceutical Formulations

High-performance liquid chromatography (HPLC) was utilized to analyze the purity and potency of processed samples. Samples were weighed and accurately transferred to 100-mL volumetric flasks to prepare 100 μg/mL solutions of BI 667. A 95:5 v/v ratio of methanol to deionized water was used as the diluent. Approximately two-thirds diluent was added to the volumetric flask and sonicated for 30 s before filling to volume. The solutions were sonicated for another 30 s and then immediately transferred to 2-mL HPLC vials for analysis.

Samples were analyzed with a Thermo Scientific Dionex UltiMate 3000 HPLC System (Thermo Scientific, Sunnyvale, Calif.). An UltiMate 3000 Autosampler was utilized to inject 10 μL samples. The HPLC system also included dual UltiMate Pumps and an UltiMate RS Variable Wavelength Detector operating at 225 nm. The aqueous mobile phase (A) consisted of 0.05% v/v TFA in deionized water, and the organic mobile phase (B) consisted of 0.05% TFA in acetonitrile. A flow rate of 1 mL/min ran isocratic from 0-5 min at 80% A, 20% B, and then a gradient was run to achieve 20% A, 80% B from 5.1-15 min. A second gradient was run to achieve 5% A, 95% B from 15.1-16 min, a third gradient was run from 16.1-17 min to achieve 80% A, 20% B, and then finally, the flow was held isocratic at 80% A, 20% B from 17.1-20 min. The 20-min injection time was sufficient to separate three potential impurities. Injections were passed through a Luna® C18(2) reversed phase column, 3.0 mm×100 mm, with 3 μm packing (Phenomenex®, Torrance, Calif.) kept at room temperature. The retention time of BI 667 was approximately 9.0 min. All analyses maintained linearity from 1-200 μg/mL. Chromeleon™ Chromatography Data System Version 7.2.9 (Thermo Scientific, Sunnyvale, Calif.) was used to process all chromatography data.

A small volume, pH-shift dissolution with biorelevant media was employed to mimic gastrointestinal transit of orally administered KSD- and SDD-processed BI 667 samples in the fasted state. Non-sink conditions were tested to evaluate amorphous BI 667's propensity to recrystallize when supersaturated in media. Dissolution was performed in a VanKel V7000 dissolution tester (Agilent Technologies, Inc., Santa Clara, Calif.) equipped with apparatus 2 (paddles) and 150 mL glass vessels operated at a temperature of 37.0±0.2° C. and a paddle speed of 100 rpm. 120 mg of processed KSD and SDD materials (40 mg equivalents BI 667) were added to vessels containing 90 mL of 0.01 N HCl. After 30 min, 60 mL of FaSSIF (2.24 g/L SIF in 0.1 M sodium phosphate buffer, pH 6.8) was added to each vessel to make a total volume of 150 mL. 800 μL samples were taken with media replacement at 5, 10, 15, 25, 35, 45, 60, 90, 120, 180, 240, and 360 min. Samples were immediately filtered through 0.22 μm, 13 mm PES syringe filters and diluted 1:1 with 95:5 methanol:deionized water. The concentration of BI 667 at each time point was measured using the aforementioned HPLC method. All dissolution samples were performed in triplicate (n=3).

Non-sink, pH-shift dissolution of all KSD species were evaluated to understand release kinetics differences between differing particle sizes (and different milling mechanisms) of thermokinetically processed material. The dissolution profiles are summarized in FIG. 9. In acidic media, all KSD particles show similar release, and a trend is observed, where increasing the particle size of the KSD material tends to decrease the release in acidic media. This parameter, C_{max, acidic}, which is the maximum concentration recorded in acidic media, as well as the ratio of C_{max, acidic}/C_{eq, neutral}, (where C_{eq, neutral} is obtained by determining the equilibrium solubility of the drug in neutral pH media, which represents that of intestinal media, e.g., pH 6.8 FaSSIF) were monitored and it was observed that increasing particle size decreases the C_{max, acidic}/C_{eq, neutral} ratio for KSD particles. These profiles are dissimilar to the physical mixture, where BI 667 rapidly springs to a concentration in solution near its equilibrium solubility. Upon addition of neutral media at t=30 min, all KSD particles exhibit an increase in BI 667 concentration. With the exception of KSD CM species, the smaller the KSD particles, the faster BI 667 springs to supersaturation. Additionally, larger KSD particles appear to have a delayed t_{max, diss} and a lower C_{max, diss} when compared with smaller KSD particles, but they tend to maintain higher levels of supersaturation for longer periods in the dissolution test. Dissolution properties (C_{max, diss}, t_{max, diss}, C_{max, acidic}, C_{max, acidic}/C_{eq, neutral} and AUDC_{0-360 min}) of all KSD species are summarized in Table 2. For KSD particles, the highest C_{max, diss} was achieved by KSD 75-125 particles, and the largest AUDC_{0-360 min} was achieved by KSD 425-600 particles.

TABLE 2
Dissolution characteristics of KSD, SDD, and physical
mixture particles in non-sink, pH-shift dissolution

| | | | | | | AUDC_{0-380 min} |
|---|---|---|---|---|---|---|
| | C_{max, diss} ± | t_{max, diss} | C_{max, acidic} ± | C_{max, acidic}/ | AUDC_{0-380 min} | Relative to |
| Sample | SD (μg/mL) | (min) | SD (μg/mL) | C_{eq, neutral} | (μg · min/mL) | Crystalline |
| SDD | 116.6 ± 2.8 | 25 | 116.6 ± 2.8 | 3.64 | 8885.7 | 1.36 |
| SDD LP | 102.5 ± 1.1 | 25 | 102.5 ± 1.1 | 3.20 | 7041.7 | 1.08 |
| SDD HP | 90.9 ± 2.8 | 25 | 90.9 ± 2.8 | 2.84 | 7222.7 | 1.10 |
| KSD CM | 45.1 ± 4.5 | 60 | 29.7 ± 3.2 | 0.93 | 7007.4 | 1.07 |
| KSD < 75 | 114.3 ± 2.7 | 45 | 33.9 ± 2.1 | 1.06 | 8490.7 | 1.30 |
| KSD 75-125 | 114.5 ± 18.3 | 45 | 25.8 ± 7.1 | 0.81 | 8663.3 | 1.32 |
| KSD 125-250 | 109.5 ± 7.8 | 60 | 20.5 ± 0.9 | 0.64 | 8468.6 | 1.29 |
| KSD 250-425 | 82.8 ± 4.3 | 90 | 13.5 ± 0.4 | 0.42 | 8444.6 | 1.29 |
| KSD 425-600 | 78.8 ± 3.7 | 90 | 9.8 ± 0.2 | 0.31 | 8788.4 | 1.34 |
| Physical Mixture | 63.1 ± 0.8 | 25 | 63.1 ± 0.8 | 1.97 | 6544.8 | — |

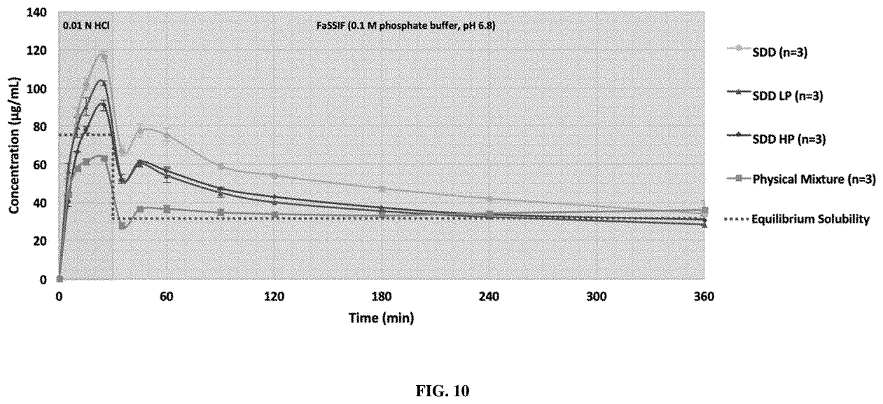

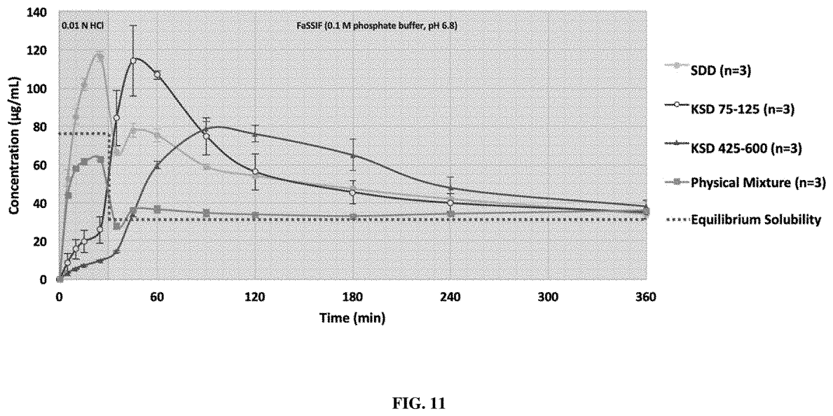

Non-sink pH-shift dissolution of all SDD species were evaluated to understand the release kinetics differences between different post-processing pressures applied during dry granulation compared with native spray dried particles. The dissolution profiles are summarized in FIG. 10. In acidic media, all spray dried particles rapidly achieve BI 667 supersaturation, and upon pH-shift, the concentrations of BI 667 in solution decrease proportional to the amount of neutral media added (i.e., the amount of BI 667 in solution is roughly equivalent, but the volume of dissolution media is increased). Notably, by increasing pressure and residence time during the slugging portion of dry granulation, the amount of BI 667 release in acidic media was modulated. Specifically, the C_{max, acidic} value decreased as the pressure applied in slugging increased. However, both SDD LP and SDD HP particles have similar dissolution profiles in neutral media. Native SDD particles exhibit the highest C_{max, diss} and AUDC_{0-360 min} compared to SDD LP and SDD HP particles. Notably, the C_{max, diss} for all SDD materials occurred in acidic media, and thus the C_{max, acidic}/C_{eq, neutral} ratio for SDD particles was substantially higher than that of KSD materials. Dissolution properties (C_{max, diss}, t_{max, diss}, C_{max, acidic}, C_{max, acidic}/C_{eq, neutral} and AUDC_{0-360 min}) of all SDD species are summarized in Table 2.

Based on observed dissolution profiles and preliminary understanding of BI 667 release kinetics in non-sink pH-shift dissolution, four species of particles were selected for in vivo oral pharmacokinetic analysis in a male Beagle dog study. The four species selected were SDD, KSD 75-125, KSD 425-600, and physical mixture particles. The dissolution profiles of the four selected particle species are summarized in FIG. 11. From this summary, it becomes apparent that each selected species predominates (maintains significantly higher BI 667 concentration) for a portion of the dissolution test. For example, SDD particles exhibit significantly higher BI 667 concentrations throughout the entirety of time in acidic media (t=0-30 min) when compared with KSD particles. These data suggest SDD particles would release a significant portion of BI 667 in gastric media during oral BI 667 administration. Conversely, two different species of KSD particles were selected for oral administration, as each exhibit significantly higher BI 667 concentrations compared with SDD particles in neutral media (though higher BI 667 concentrations take place at different times of the test), which suggests the KSD particles would release more BI 667 in the intestinal phase during oral administration. KSD 75-125 particles exhibit higher BI 667 concentrations (compared with SDD particles) from 35-120 min, while KSD 425-600 particles exhibit higher BI 667 concentrations from 90-360 min. Interestingly, the relative AUDC_{0-360 min} compared with the BI 667 physical mixture of the selected particles are essentially identical; 1.36×, 1.32×, and 1.34× for SDD, KSD 75-125, and KSD 425-600, respectively. However, the C_{max, acidic}/C_{eq, neutral} ratio for the three species varied, where SDD material was more similar to the physical mixture C_{max, acidic}/C_{eq, neutral} ratio, while KSD material maintained a C_{max, acidic}/C_{eq, neutral} ratio below 1. Therefore, by selecting three species with similar relative AUDC values but differing C_{max, acidic}/C_{eq, neutral} ratios, where BI 667 supersaturation is most important for enhanced oral bioavailability was evaluated.

### Example 5: Dissolution-Permeation Testing of BI 667 Pharmaceutical Formulations with Pion μFLUX Apparatus

In vitro dissolution-permeation studies were conducted using a Pion μFLUX apparatus (Pion Inc., Boston, Mass.). The apparatus consisted of a 20 mL donor and 20 mL acceptor compartment, which were separated by an artificial membrane (PVDF, 0.45 μm, 8.55 cm²) impregnated with 25 μL of Pion GIT-0 lipid (20% w/w phospholipid dissolved in dodecane). In the case of oral delivery, the donor compartment represented gastrointestinal media and the artificial membrane represented the intestinal wall, while the acceptor chamber represented the intravascular system (blood circulation).

In the donor compartment, 16 mg of KSD- and SDD-processed samples were accurately weighed and added prior to the addition of donor media. Care was taken to ensure the sink index of BI 667 to dissolution media between the non-sink dissolution and the μFLUX™ apparatus were kept consistent. In the donor compartment, 12 mL 0.01 N HCl was added following the first collected spectra at t=0 min. This volume represented a sufficient volume to completely saturate the membrane, and the pH-shift method (3 parts acidic media, 2 parts neutral media) was consistent with the non-sink dissolution. At t=30 min, 8 mL FaSSIF in pH 6.8 phosphate buffer was added to the donor chamber. In the acceptor compartment, 20 mL Acceptor Sink Buffer (ASB), (Pion Inc.) was added prior to collection of the first spectra. Both the donor and acceptor compartments were stirred at 100 rpm with cross stir bars. Dissolution media was kept at 37.0±0.2° C. by circulating water through a Julabo Corio™ CD immersion circulator (Julabo USA, Inc., Allentown, Pa.) mounted to a μDiss Profiler™ (Pion Inc.) for all experiments.

BI 667 absorbance in both the donor and acceptor compartments were collected by UV probes (2 mm path length) between wavelengths 330 and 340 nm via a Rainbow UV spectrometer (Pion Inc.). BI 667 absorbance was collected every 5 min from 0-360 min, and the concentration of BI 667 in the donor and acceptor compartments were calculated from AuPRO™ v5.1 software (Pion Inc.). Reported BI 667 concentrations represent the average of two samples (n=2).

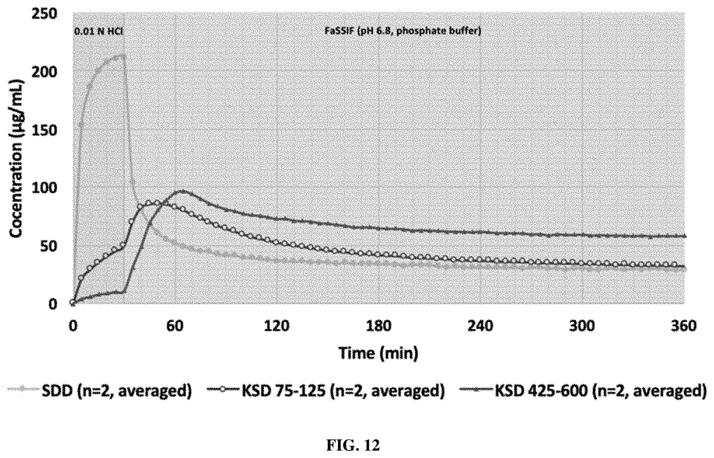

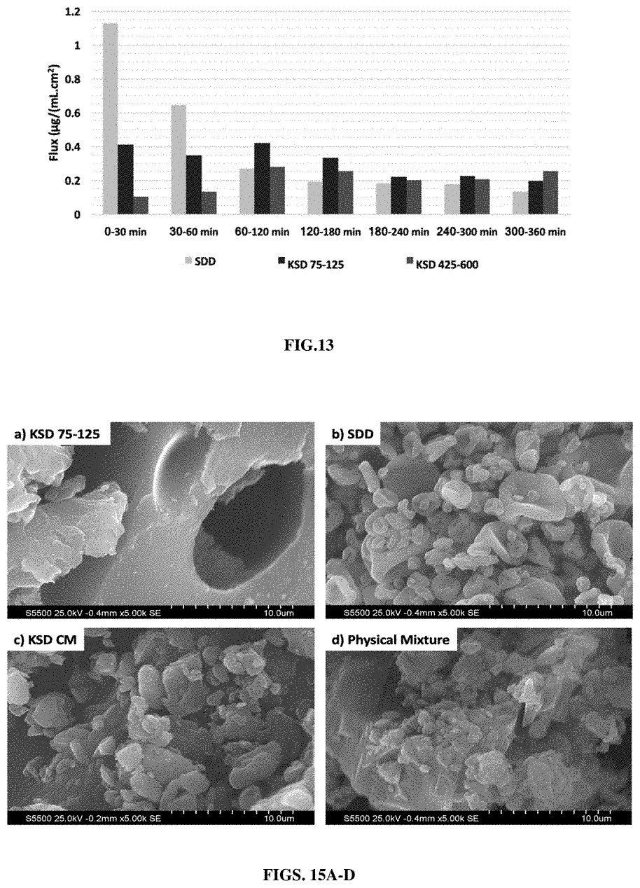

To gain a further understanding of the particles to be dosed in the in vivo Beagle dog model, flux was calculated using a Pion μFLUX™ apparatus. The data obtained from the apparatus is presented in FIG. 12 and FIG. 13, where FIG. 12 represents BI 667 concentrations in the donor compartment throughout the entirety of the test, and FIG. 13 represents the calculated flux as a result of BI 667 concentrations in the acceptor compartment during different time portions of the test. The dissolution curve achieved in the donor compartment is similar to that of the pH-shift dissolution apparatus with one exception; Dissolution of BI 667 in the SDD is substantially heightened in acidic media. Flux of BI 667 across the membrane follows the trend observed during the donor compartment dissolution; Early stage (the acidic stage and early neutral stage) flux of BI 667 in SDD particles is substantially higher than that of the KSD particles. However, as BI 667 concentrations for KSD particles maintain higher levels of supersaturation later in the dissolution apparatus, the rate of flux of these species supersedes the rate of flux of BI 667 in the SDD particles after 60 min. Interestingly, the rate of flux of KSD 75-125 particles is greater than KSD 425-600 particles (aside from 300-360 min) even when KSD 425-600 particles maintain a higher concentration throughout the neutral portion of the test.

### Example 6: Pharmacokinetic Testing in Male Beagle Dogs of BI 667

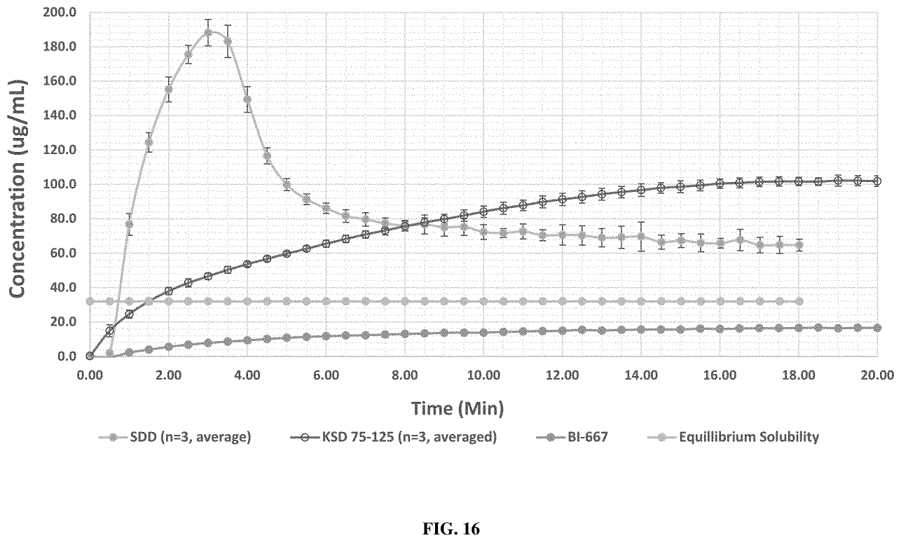

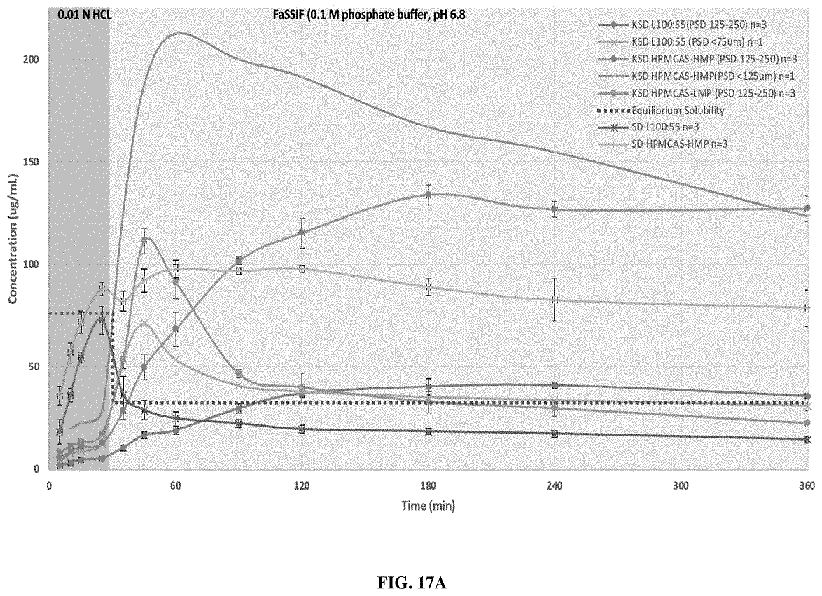

In vivo non-crossover oral pharmacokinetic analysis in a fasted male Beagle dog model was conducted at Pharmaron (Ningbo, China). Samples were suspended in a 0.5% methylcellulose/0.1% Tween vehicle at a concentration of 12 mg/mL (BI 667 concentration 4 mg/mL). A 1.33 mg/mL suspension was prepared in same vehicle and tested in a PION dissolution apparatus as shown in FIG. 16, which mimics the preparation conditions used. A target dose of 20 mg/kg BI 667 was administered orally in each arm of the study. Each arm consisted of four dogs ranging in weights between 8 and 14 kg. Dogs were fasted overnight (minimum 12 hours) prior to dosing, and food was returned 4 hours after dose administration. Following dosage administration, 10 mL purified water was administered to each dog to flush out the remaining contents in the syringe. Approximately 1 mL of blood was collected by venipuncture for each time point. A pre-dose blood sample was collected, and samples were collected at t=0.25, 0.5, 1, 2, 4, 6, 8, 12, and 24 hr. Sodium heparin was utilized as an anticoagulant and blood samples were centrifuged at ˜2,000 g (force) for 10 min at 2-8° C. to obtain plasma. BI 667 concentration in plasma was analyzed by LC-MS/MS.

Pharmacokinetic parameters for the male Beagle dog study were calculated using Phoenix™ WinNonlin v6.1 (Certara USA, Inc., Princeton, N.J.). The following parameters were calculated following oral administration of BI 667-containing samples: Cₘₐₓ, Tₘₐₓ, AUC₀₋₂₄ hr, T_{1/2}, AUC_{0-∞}. A paired two-tailed t-test with an alpha value of 0.05 was conducted for the male Beagle dog study to assess for statistically significant differences in plasma concentrations between the different formulations.

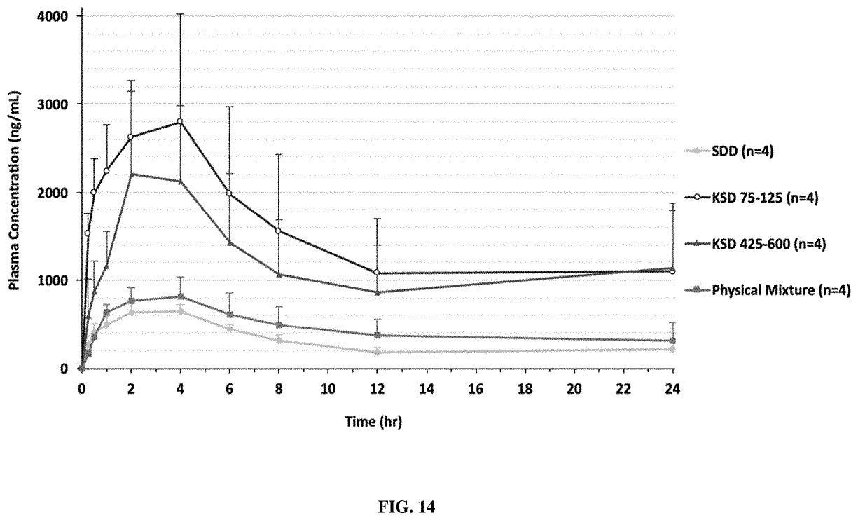

Following oral administration of BI 667 in four particle species (SDD, KSD 75-125, KSD 425-600, and physical mixture) in male Beagle dogs, plasma BI 667 concentrations were evaluated, and the data are summarized in FIG. 14. Both KSD particle species exhibited higher Cₘₐₓ values and maintained higher plasma levels for 24 hours when compared with both the SDD and physical mixture particles. Additionally, KSD 75-125 particles' plasma concentrations maintained statistically significant (p<0.05) higher plasma concentrations than SDD and physical mixture particles until 6 and 4 hours, respectively.

Table 3 summarizes the calculated pharmacokinetic parameters from the plasma concentration data. The KSD 75-125 particles outperformed all other tested particle species and provided the most BI 667 exposure in the Beagle dog study. The observed Cₘₐₓ and AUC₀₋₂₄ hr values were 3.6 and 3.1× the physical mixture (i.e., crystalline BI 667) particles, respectively. Similar to the in vitro dissolution, the tₘₐₓ of the larger KSD particles (KSD 425-600) is later than that of the smaller particles (KSD 75-125). Surprisingly, the SDD particles were outperformed by the physical mixture particles (though not statistically significant), and demonstrated an AUC_{0-24 hr} 0.6× the physical mixture.

TABLE 3
Pharmacokinetic values after oral administration of BI 667, KSD 75-
125, KSD 425-600, and physical mixture particles in male Beagle dogs

| | Dose | Cₘₐₓ | tₘₐₓ | AUC_{0-24 hr} | AUC_{0-24 hr}/Dose | AUC_{0-24 hr} Relative |
|---|---|---|---|---|---|---|
| Sample | (mg/kg) | (ng/mL) | (h) | (ng · hr/mL) | (mg · hr/mL) | to Crystalline |
| SDD | 20 | 670 ± 106 | 3.5 ± 1.0 | 74954 ± 2093 | 375 ± 105 | 0.63 |
| KSD 75-125 | 20 | 2963 ± 1137 | 2.0 ± 1.4 | 36,239 ± 15,423 | 1812 ± 771 | 3.07 |
| KSD 425-600 | 20 | 2215 ± 943 | 2.5 ± 1.0 | 28,699 ± 13,809 | 1435 ± 690 | 2.43 |
| Physical Mixture | 20 | 824 ± 219 | 3.0 ± 1.2 | 11,804 ± 5379 | 590 ± 269 | — |

### Example 7: Analysis of Particle Morphology, Size, and Surface Area

Scanning electron microscopy (SEM) was utilized to visualize particle differences between the samples dosed in the in vivo dog study. Samples were imaged with a Hitachi S5500 SEM/STEM (Hitachi, Krefeld, Germany). The samples were placed on double-sided carbon adhesive tape, mounted on standard aluminum SEM stubs, and sputter coated with gold/palladium (Au/Pd) 60/40 using an Electron Microscopy Sciences 500× sputter coater (Electron Microscopy Sciences, Hatfield, Pa.). The sputter coater operated at 40 mA and a sputtering time of 45 s. The samples were evaluated at a nominal magnification of 5,000× at high vacuum, and the incident electron beam was set at 25 kV.

The surface structures of KSD, SDD, KSD CM, and physical mixture particles were imaged with SEM. FIGS. 15A-D show visualized surface characteristics of the different particle species generated in the study. FIG. 15A depicts KSD 75-125 particles. Small cavities are found amongst the particles, but overall, the thermokinetically compounded particles are smooth and uniform in appearance. In many instances, some smaller particles (i.e. <75 μm particles) are adhered to the particles imaged. These findings are consistent with all KSD species collected on the series of sieves (data not shown). FIG. 15B depicts SDD particles. The structure of SDD particles appear toroidal, smooth, and contain crevices and folds that increase the SSA of the particles. FIG. 15C depicts KSD CM material, the particles contain a wide range of small particle sizes, and the particles maintain smooth surfaces similar to the KSD 75-125 material. FIG. 15C depicts physical mixture particles, where BI 667 is adhered to the surface of larger HPMCAS-MMP particles.

The particle size distributions (PSDs) were analyzed with a Mastersizer 3000E using a Hydro EV wet stage dispersion unit (Malvern Panalytical, Malvern, UK) using Mie scattering theory. Acquisition was conducted with a 300-mm lens using a red laser. Deionized water was used as the dispersant and agitation of the dispersant was provided by ultrasound and a propeller speed of 2,400 rpm. Background and sample measurement durations were both set to 10 s, and 30 measurements were taken for each sample. Sample material was added to the dispersant until obscuration of the laser was >2%, and samples were taken in the range of 0.1-25% obscuration. Mastersizer 3000 Software v3.62 (Malvern Panalytical, Malvern, UK) was utilized to obtain the D10, D50, and D90 for all samples analyzed. Sample PSDs are reported as the average of 5 measurements (n=5).

The specific surface area (SSA) of samples were determined with a single-point Brunauer-Emmett-Teller (BET) method using a Monosorb® Rapid Surface Area Analyzer, MS-25 (Quantachrome, Boynton Beach, Fla.). The samples were accurately weighed to approximately 200 mg and added to a tared glass sample holder. The samples were allowed to outgas for 24 h at 40° C. under dry helium dioxide. BET nitrogen adsorption and desorption was performed using a 30% v/v mixture of nitrogen in helium. SSA values were determined from desorption of nitrogen. All samples were run in triplicate.

The D10, D50, and D90 of all species of KSD, SDD, and physical mixture were evaluated and are shown in Table 4. Of note, the SDD HP particles were the only species that exhibited a bimodal PSD. The measured SSAs of all the aforementioned species were also included on Table 4.

TABLE 4
Particle size distribution and specific
surface area of the generated particles

| | D10 | D50 | D90 | Specific Surface |
|---|---|---|---|---|
| Sample | (μm) | (μm) | (μm) | Area ± SD (m²/g) |
| SDD | 3.5 | 6.1 | 11.2 | 3.37 ± 0.08 |
| SDD LP | 2.7 | 5.4 | 13.1 | 3.56 ± 0.08 |
| SDD HP | 3.5 | 9.9 | 74.5 | 3.01 ± 0.09 |
| KSD CM | 4.2 | 16.3 | 41.9 | 1.52 ± 0.04 |
| KSD < 75 | 29.9 | 57.0 | 97.4 | 0.34 ± 0.04 |
| KSD 75-125 | 44.5 | 102 | 177 | 0.34 ± 0.06 |
| KSD 125-250 | 135 | 264 | 488 | 0.28 ± 0.04 |
| KSD 250-425 | 219 | 380 | 579 | 0.21 ± 0.03 |
| KSD 425-600 | 409 | 578 | 648 | 0.17 ± 0.05 |
| Physical Mixture | 11.0 | 60.1 | 200 | 2.68 ± 0.04 |

### Example 8: Pharmaceutical Formulations of the Present Disclosure Having any Pharmaceutically Acceptable Weakly Basic Drug and an Ionic Polymer

It is contemplated that the exemplary formulations described in the Examples herein having BI 667 and HPMCAS-MMP will show the same or similar results in terms of pharmacokinetics and therapeutic effects. Accordingly, it is contemplated that a weakly basic drug and an ionic polymer will show the same or similar results in terms of pharmacokinetics and therapeutic effects. Further, it is contemplated that formulations of a weakly basic drug and an ionic polymer having a specific surface area between 0.05 m²/g and 2 m²/g will show the same or similar results in terms of pharmacokinetics and therapeutic effects.

### Example 9: Dissolution Testing of BI-667 Pharmaceutical Formulations with Different Ionic Polymers

Different ionic polymers were selected to demonstrate feasibility outside of the system studied. HPMCAS-HMP, LMP and Eudragit L 100-55 were evaluated as additional ionic polymers.

High-performance liquid chromatography (HPLC) was utilized to analyze the purity and potency of processed samples. Samples were weighed and accurately transferred to 100-mL volumetric flasks to prepare 100 μg/mL solutions of BI 667. A 95:5 v/v ratio of methanol to deionized water was used as the diluent. Approximately two-thirds diluent was added to the volumetric flask and sonicated for 30 s before filling to volume. The solutions were sonicated for another 30 s and then immediately transferred to 2-mL HPLC vials for analysis.

Samples were analyzed with a Thermo Scientific Dionex UltiMate 3000 HPLC System (Thermo Scientific, Sunnyvale, Calif.). An UltiMate 3000 Autosampler was utilized to inject 10 μL samples. The HPLC system also included dual UltiMate Pumps and an UltiMate RS Variable Wavelength Detector operating at 225 nm. The aqueous mobile phase (A) consisted of 0.05% v/v TFA in deionized water, and the organic mobile phase (B) consisted of 0.05% TFA in acetonitrile. A flow rate of 1 mL/min ran isocratic from 0-5 min at 80% A, 20% B, and then a gradient was run to achieve 20% A, 80% B from 5.1-15 min. A second gradient was run to achieve 5% A, 95% B from 15.1-16 min, a third gradient was run from 16.1-17 min to achieve 80% A, 20% B, and then finally, the flow was held isocratic at 80% A, 20% B from 17.1-20 min. The 20-min injection time was sufficient to separate three potential impurities. Injections were passed through a Luna® C18(2) reversed phase column, 3.0 mm×100 mm, with 3 μm packing (Phenomenex®, Torrance, Calif.) kept at room temperature. The retention time of BI 667 was approximately 9.0 min. All analyses maintained linearity from 1-200 μg/mL. Chromeleon™ Chromatography Data System Version 7.2.9 (Thermo Scientific, Sunnyvale, Calif.) was used to process all chromatography data.

A small volume, pH-shift dissolution with biorelevant media was employed to mimic gastrointestinal transit of orally administered KSD- and SDD-processed BI 667 samples in the fasted state. Non-sink conditions were tested to evaluate amorphous BI 667's propensity to recrystallize when supersaturated in media. Dissolution was performed in a VanKel V7000 dissolution tester (Agilent Technologies, Inc., Santa Clara, Calif.) equipped with apparatus 2 (paddles) and 150 mL glass vessels operated at a temperature of 37.0±0.2° C. and a paddle speed of 100 rpm. 120 mg of processed KSD and SDD materials (40 mg equivalents BI 667) were added to vessels containing 90 mL of 0.01 N HCl. After 30 min, 60 mL of FaSSIF (2.24 g/L SIF in 0.1 M sodium phosphate buffer, pH 6.8) was added to each vessel to make a total volume of 150 mL. 800 μL samples were taken with media replacement at 5, 10, 15, 25, 35, 45, 60, 90, 120, 180, 240, and 360 min. Samples were immediately filtered through 0.22 μm, 13 mm PES syringe filters and diluted 1:1 with 95:5 methanol:deionized water. The concentration of BI 667 at each time point was measured using the aforementioned HPLC method. All dissolution samples were performed in triplicate (n=3), unless otherwise stated.

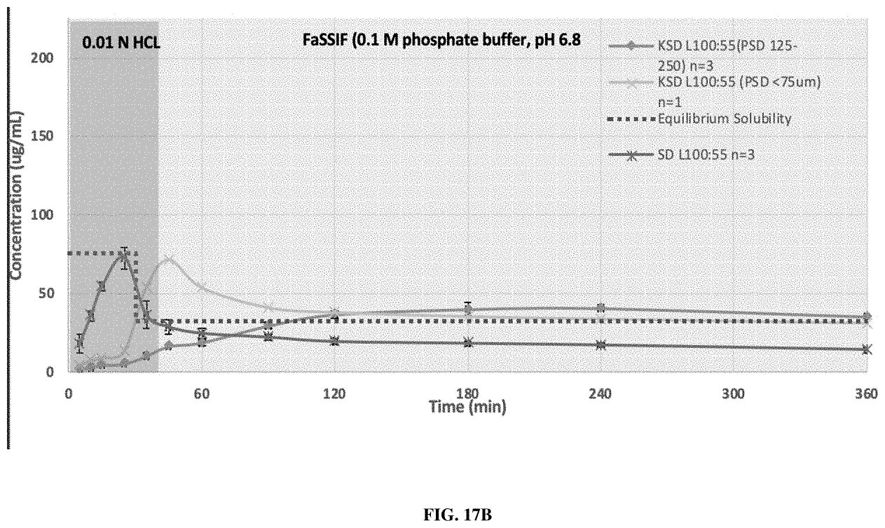

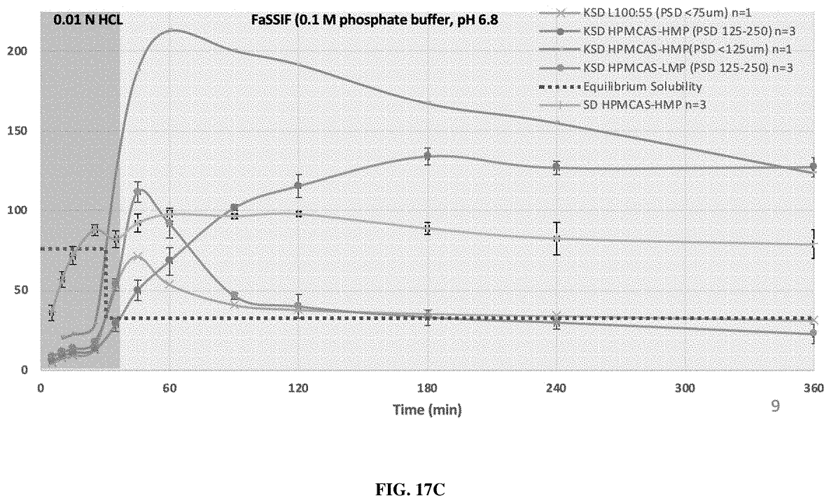

Non-sink, pH-shift dissolution of all KSD species were evaluated to understand release kinetics differences between differing particle sizes (and different milling mechanisms) of KinetiSol®-produced material. The dissolution profiles are summarized in FIGS. 17A-C. In acidic media, all KSD particles show similar release, and a trend is observed, where increasing the particle size of the KSD material tends to decrease the release in acidic media. We monitor this parameter, C_{max, acidic}, which is the maximum concentration recorded in acidic media, as well as the ratio of C_{max, acidic}/C_{eq, neutral}, where increasing particle size decreases the C_{max, acidic}/C_{eq, neutral} ratio for KSD particles. These profiles are dissimilar to the physical mixture, where BI 667 rapidly springs to a concentration in solution near its equilibrium solubility. Upon addition of neutral media at t=30 min, all KSD particles exhibit an increase in BI 667 concentration. The smaller the KSD particles, the faster BI 667 springs to supersaturation. Additionally, larger KSD particles appear to have a delayed t_{max, diss} and a lower C_{max, diss} when compared with smaller KSD particles. Dissolution properties (C_{max, diss}, t_{max, diss}, C_{max, acidic}, C_{max, acidic}/C_{eq, neutral} and AUDC_{0-360 min}) of all KSD species are summarized in Table 5. For KSD particles, the highest C_{max, diss} was achieved by KSD<125 particles.

TABLE 5

| | C_{max, diss} ± SD | T_{max, diss} | C_{max, acidic} ± SD | C_{max, acidic}/ |
|---|---|---|---|---|
| Sample | (ug/mL) | (min) | (ug/mL) | C_{eq, neutral} |
| SDD- HMP | 97.6 ± 4.3 | 60 | 88.0 ± 3.33 | 2.75 |
| SDD- L100-55 | 72.6 ± 6.8 | 25 | 72.6 ± 6.8 | 2.27 |
| KSD L100-55 < 75 n = 1 | 71.1 | 45 | 12.9 | 0.40 |
| KSD L100-55 125-250 | 40.7 ± 1.3 | 240 | 5.4 ± 0.5 | 0.17 |
| KSD-HMP < 125 n = 1 | 212.8 | 60 | 28.42 | 0.89 |
| KSD-HMP 125-250 | 134.0 ± 5.1 | 180 | 12.7 ± 0.9 | 0.40 |
| KSD-LMP 125-250 | 111.6 ± 6.1 | 45 | 17.0 ± 1.3 | 0.53 |

The dissolution profiles are summarized in FIGS. 17A-C. In acidic media, all spray dried particles rapidly achieve BI 667 supersaturation, and upon pH-shift, the concentrations of BI 667 in solution decrease proportional to the amount of neutral media added (i.e., the amount of BI 667 in solution is roughly equivalent, but the volume of dissolution media is increased). Dissolution properties (C_{max, diss}, t_{max, diss}, C_{max, acidic}, C_{max, acidic}/C_{eq, neutral} and AUDC_{0-360 min}) of all SDD species are summarized in Table 5.

The above disclosure contains various examples of pharmaceutical formulations, final solid dosage forms, methods of forming pharmaceutical formulations, and methods of administering pharmaceutical formulations. Aspects of these various examples may all be combined with one another, even if not expressly combined in the present disclosure, unless they are clearly mutually exclusive. For example, a specific pharmaceutical formulation may contain amounts of components identified more generally or may be administered in any way described herein.

In addition, various example materials are discussed herein and are identified as examples, as suitable materials, and as materials included within a more generally-described type of material, for example by use of the term “including” or “such-as.” All such terms are used without limitation, such that other materials falling within the same general type exemplified but not expressly identified may be used in the present disclosure as well.

In addition, unless it is clear that a precise value is intended, numbers recited herein should be interpreted to include variations above and below that number that may achieve substantially the same results as that number, or variations that are “about” the same number.

Finally, a derivative of the present disclosure may include a chemically modified molecule that has an addition, removal, or substitution of a chemical moiety of the parent molecule.

The above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

The present disclosure may be better understood through reference to the following claims, which are intended to form part of this Specification in the same manner as the preceding text, and which may be combined with one another and with other portions of this Specification in any fashion and combinations, unless such combinations are clearly mutual exclusive.

1. A pharmaceutical formulation comprising:
a weakly basic drug; and
an ionic polymer excipient, together in an amorphous solid dispersion.

2. The pharmaceutical formulation of claim 1, wherein the ionic polymer excipient comprises hypromellose acetate succinate

3. The pharmaceutical formulation of claim 1, wherein the ionic polymer excipient is selected from the group consisting of hydroxypropyl methylcellulose acetate succinate, polyvinyl acetate phthalate, hypromellose acetate succinate, hydroxypropyl methylcellulose phthalate, methacylic acid-co-ethyl acrylate, methacylic acid-co-methyl methacrylate; and combinations thereof.

4. The pharmaceutical formulation of claim 1, wherein the weakly basic drug and ionic polymer are present in a weight ratio of between 1:0.25 to 1:50, inclusive.

5. The pharmaceutical formulation of claim 1, wherein the amorphous solid dispersion is made up of particles, wherein the average specific surface area of the particles is less than 2.0 (m²/g), inclusive, such as wherein the particles of the amorphous solid dispersion have a specific surface area of greater than 0.05 (m²/g), inclusive.

6. (canceled)

7. The pharmaceutical formulation of claim 1, wherein the weakly basic drug comprises a primary, secondary or tertiary amine functional group.

8. The pharmaceutical formulation of claim 1, wherein the weakly basic drug is selected from the group consisting of BI 639667, ciprofloxacin, mitoxantrone, epirubicin, daunorubicin, doxorubicin, vincristine, vinblastine, lidocaine, chlorpromazine, dibucaine, propranolol, timolol, quinidine, pilocarpine, physostigmine, dopamine, serotonin, imipramine, diphenhydramine, quinine, chloroquine, quinacrine, ritonavir, itraconazole, posaconazole, nevirapine, aprepitant, albendazole, mebendazole, amprenavir, abiraterone, saquinavir, rifabutin, anthracyclines, vinca alkaloids, lamivudine, zalcitabine, didanosine, efavirenz, zidovudine, nelfinavir, indinavir, chloroquine, azathioprine, atazanavir, amiodarone, terfenadine, tamoxifen, velpatasvir, elbasvir and codeine, pharmaceutically acceptable salts thereof, and combinations thereof.

9. The pharmaceutical formulation of claim 1, wherein a non-sink, pH-shift dissolution test of the pharmaceutical formulation has a C_{max, acidic}/C_{eq, neutral ratio} less than or equal to 1.10.

10. A method of forming a pharmaceutical formulation, the method comprising compounding a weakly basic drug and a ionic polymer excipient in a thermokinetic mixer at a temperature less than or equal to 200° C. for less than 300 seconds to form an amorphous solid dispersion of a weakly basic drug and an ionic polymer.

11. The method of claim 10, wherein the pharmaceutical formulation is a pharmaceutical formulation comprising a weakly basic drug and an ionic polymer excipient, together in an amorphous solid dispersion.

12. The method of claim 10, wherein compounding in the thermokinetic mixer does not cause more than 20% of the weakly basic drug to thermally degrade.

13. A method of forming a pharmaceutical formulation, the method comprising melt processing a weakly basic drug and an ionic polymer excipient to form an amorphous solid dispersion of the weakly basic drug and the ionic polymer excipient in which less than 20% of the weakly basic drug thermally degrades.

14. The method of claim 13, wherein the pharmaceutical formulation is a pharmaceutical formulation comprising a weakly basic drug and an ionic polymer excipient, together in an amorphous solid dispersion.

15. A method of administering a weakly basic drug, the method comprising orally delivering to a patient, with a stomach having stomach contents, a small intestine having small intestine contents, and blood plasma, a pharmaceutical formulation of claim 1.

16. The method of claim 15, wherein at least 50%, inclusive, of the weakly basic drug dissolves in the small intestine of the patient.

17. The method of claim 15, wherein between 0.05% and 30%, inclusive, of the weakly basic drug is dissolved in the stomach of the patient.

18. The method of claim 15, wherein the weakly basic drug does not reach a saturation concentration in the stomach contents of the patient.

19. The method of claim 15, wherein the weakly basic drug does reach a saturation concentration in the small intestine contents of the patient.

20. The method of claim 15, wherein the weakly basic drug reaches a max concentration level of greater than or equal to 1800 ng/mL in the blood plasma.

21. The method of claim 15, wherein the weakly basic drug has a AUC_{0-24 hr} value of greater than or equal to 20,000 (ng×hr)/mL in the blood plasma.
